(19) United States
(12) Patent Application Publication
(10) Pub. No.: US 2003/0225431 A1
(43) Pub. Date: Dec. 4, 2003

(54) Needles for Korean traditional medical treatment

(76) Inventor: Tae woo Yoo, Seoul (KR)

Correspondence Address: Richard M. Goldberg, Suite 419, 25 East Salem Street, Hackensack, NJ, 07601, US

(21) Appl. No.: 10/207,467

(22) Filed: Jul. 29, 2002

(30) Foreign Application Priority Data
Jun. 3, 2002 (KR) U2002-0016960

(51) Int. Cl. A61B 17/34

(52) U.S. Cl. 606/189

(57) ABSTRACT

A needle for Korean traditional medical treatment includes a grip part and a needle body, the bottom portion and the end of the needle body being coated with gold so that the needle can be applied smoothly without pain because the end of the needle body is coated with gold. Since gold is highly conductive and can control electric current of the body without side effects, the coated gold provides the effects of dispersing excess electric current at the fever, pain and inflammation parts and of inducing the electric current into functional disorders, paralysis, blood circulation disorders, shiver and convulsion parts and thus, results in medical treatment effect via functional improvement.

### BACKGROUND OF THE INVENTION

The present invention relates to needles for Korean traditional medical treatment and more particularly, to needles for Korean traditional medical treatment comprising a grip part and a needle body. The bottom portion and the end portion of the needle body are coated with gold. An end of the conventional needle body is usually uneven or jagged. However, since the end of the needle body according to the present invention is coated with gold, the needle of the present invention can be applied smoothly without pain. Gold is highly conductive and can control electric current of the body without side effects. Thus, the coated gold provides the effects of dispersing excess electric current at the fever, pain and inflammation parts and of inducing the electric current into functional disorders, paralysis, blood circulation disorders, shiver and convulsion parts and thus, results in medical treatment effect via functional improvement.

The heart generates minute electric current of a level of 10 μA and supplies it into the whole body. This current is called the body electric current.

If the whole body is equally charged with electric current, it is good for health but, if not, the body gets sick.

When electric current is excessive in the body, fever, pain and inflammation break out. When the body electrical current is insufficient, functional disorders, paralysis, blood circulation disorders, shiver and convulsion, etc. occur.

Electronic needles are used for the medical treatment of disorders of the body electrical current. Since these needles make use of 100 to 800 μA electrical current, they provide a quick treatment effect but cause vertigo, fatigue, slight fever, paralysis, hallucination, impotence, etc.

Also, in order to control body electric current, a gold needle treatment has been used as a conventional method. In this method, the gold needle is built inside of the skin. This method is very dangerous, provides side effects and is not practical due to its high price.

In addition, a conventional long needle in hand acupuncture has a pointed end which is cut sharply, but its surface is rugged or jagged. This rugged surface stimulates sensitive acupuncture points or affected parts but causes unfavorable pain.

### SUMMARY OF THE INVENTION

The present invention was designed to solve the aforementioned problems. An object of the present invention is to provide a needle for Korean traditional medical treatment which can reduce pain when applied and control the electrical current of the body without causing any side effects. The needle of the present invention comprises a grip part and a needle body. The bottom portion and the end of the needle body are coated with gold. An end of the conventional needle body is usually uneven due to insufficient grinding. However, since the end of the needle body according to the present invention is coated with gold, the needle of the present invention can be applied smoothly without causing pain. Gold is highly conductive and can control electric current of the body without side effects. Thus, the coated gold provides the effects of dispersing excess electric current at the fever, pain and inflammation parts and of inducing the electric current into functional disorders, paralysis, blood circulation disorders, shiver and convulsion parts and thus, results in medical treatment effect via functional improvement.

### BRIEF DESCRIPTION OF THE DRAWINGS

The other objects and features of the present invention will be hereinafter explained in detail with reference to the accompanying drawings.

FIG. 1 is a perspective view of the present invention.

FIG. 2 is a cross-sectional view of the present invention.

FIG. 3 is an enlarged cross-sectional view of the part A of FIG. 2

FIG. 4 is a perspective view showing an acupuncture operation.

FIG. 5 is a partial enlarged cross-sectional view of another embodiment.

FIG. 6 is an enlarged cross-sectional view of the part B of FIG. 5.

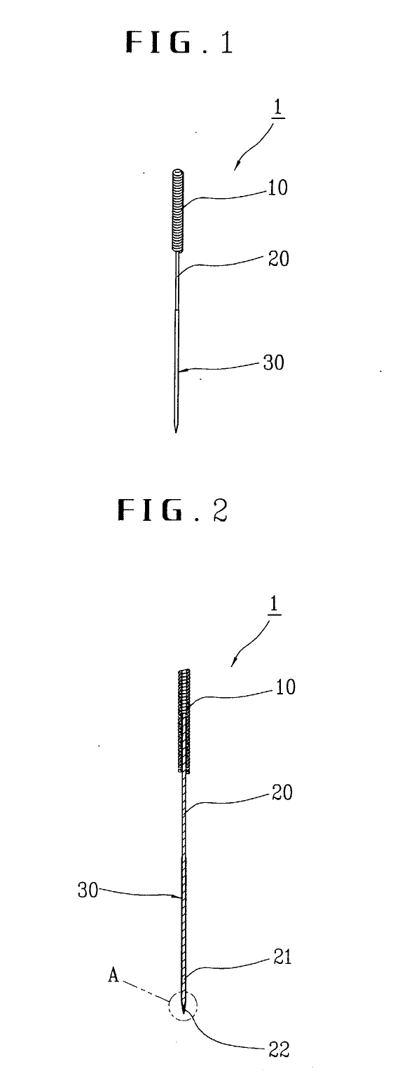

### DETAILED DESCRIPTION OF THE INVENTION

The needle 1 for Korean traditional medical treatment according to the present invention comprises a grip part 10 and a needle body 20. The bottom portion 21 and the rugged or jagged portion 23 in the end 22 are coated with gold and consist of the gold coated part 30.

Unexplained number 100 refers to the skin.

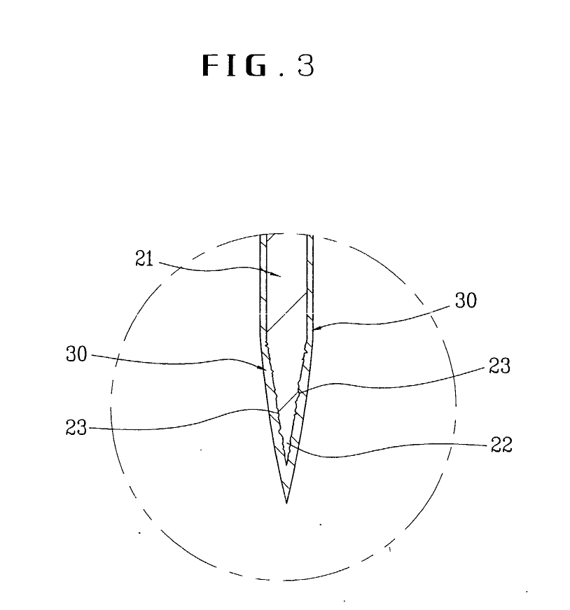

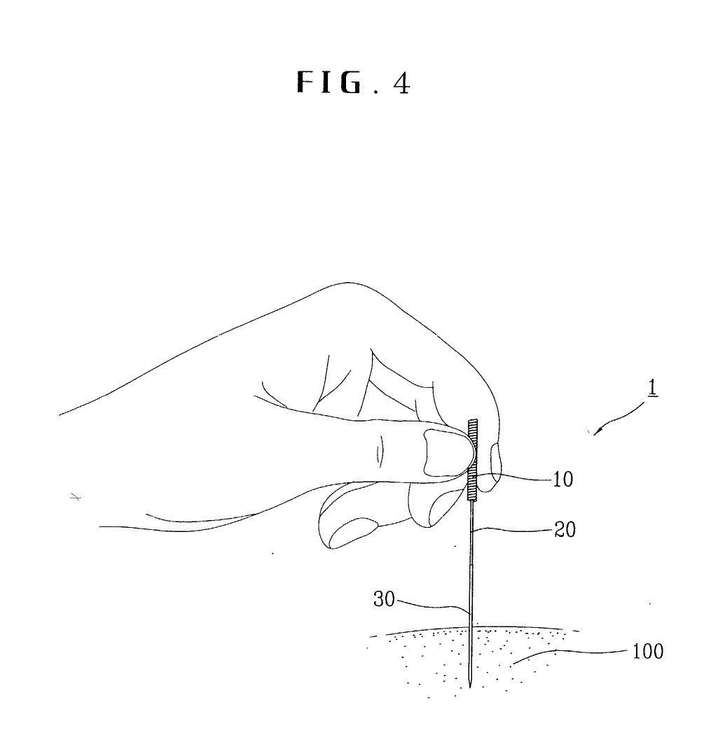

Needle 1 of the present invention is coated with gold from pointed end 22 to the bottom portion 21 which is the portion to be inserted into the skin 100. When a needle 1 is manufactured, the surface of pointed end 22 is rugged or jagged due to the shortage of grinding technology. In order to prevent this jagged end from causing pain when applied, the gold coated portion is made as illustrated in FIG. 3. As illustrated in FIG. 4, the coated portion 30 with gold is smoothly applied into acupuncture points on the skin 100 and thereby pain can be reduced.

Generally, gold has been used as medicine in Korean traditional medical treatment. For example, a pill coated with a thin golden film is taken. Powdered gold is also taken because it is good for the internal organs and mental stabilization. Specifically, gold is a highly conductive material and thus is necessary to control electric current of the body. Therefore, the needle of the present invention is designed to control electric current at the fever, pain and inflammation parts, the functional disorders, paralysis, blood circulation disorders, shiver and convulsion parts by applying the gold coated portion 30 of the needle body 20 into acupuncture spots on the skin 100.

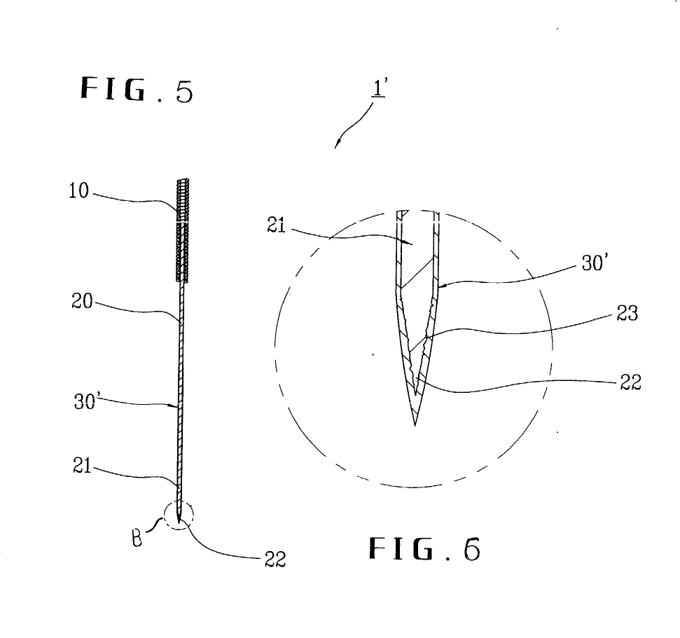

FIGS. 5 and 6 show another embodiment of a needle 1′ for Korean traditional medical treatment comprising a grip part 10 and a needle body 20. The bottom portion 21 and the end 22 of the needle body 20 are coated with silver. The needle 1′ of this embodiment can be applied smoothly without causing pain and has a high heat conductivity due to the silver coating.

If heat conductivity of silver is regarded as 100, copper is 94, gold is 53.2, iron is 11.6 and platinum is 8.2. Therefore, the heat conductivity of silver is higher than other metals. Due to this property, silver plays a role to absorb body heat and emit the heat outside when contacted with the skin. Furthermore, as silver has a strong property to absorb toxic material, it has been used to detect or absorb toxic materials.

Thus, silver can absorb overflowing energy in the acupuncture and emit it outside, and thereby, balance the energy of the body.

As silver is cheaper than gold, the needle 1′ of the present invention is economical.

As mentioned above, the needles 1 of the present invention comprise gold coated on the bottom portion 21 and the end of the needle body 22. An end of the conventional needle body is usually uneven or jagged. However, since the end of the needle body according to the present invention is coated with gold, the needle of the present invention can be applied smoothly without pain. Gold is highly conductive and can control electric current of the body without side effects. Thus, the gold coated needles of the present invention provide the effects of dispersing excess current at the fever, pain and inflammation parts and of inducing the electric current into functional disorders, paralysis, blood circulation disorders, shiver and convulsion parts, and thereby, provide a medical treatment effect.

The needles 1′ of the present invention can have the bottom portion 21 and the end 22 of the needle body 20 coated with silver. This needle 1′ can be applied smoothly without causing pain. Since silver has a higher heat conductivity than other metals, the silver coated needles 1′ of the present invention play a role to absorb body heat and emit it outside when contacted with the skin. Thereby, the silver coated needle has an effect of making the energy of the body balance.

What is claimed is:

1. A needle for Korean traditional medical treatment comprising:
a grip part, and
a needle body, said needle body having a bottom portion and a jagged portion at an end, both portions being coated with gold.

2. A needle for Korean traditional medical treatment comprising:
a grip part, and
a needle body, said needle body having a bottom portion and a jagged portion at an end, both portions being coated with silver.
